(19) United States
(12) Patent Application Publication
(10) Pub. No.: US 2025/0322310 A1
(43) Pub. Date: Oct. 16, 2025

(54) APPARATUS FOR EVALUATING ETHICALITY OF ARTIFICIAL INTELLIGENCE MODEL ACCORDING TO USER TYPE

(71) Applicant: Manki MOON, Yangju-si (KR)

(72) Inventor: Manki MOON, Yangju-si (KR)

(21) Appl. No.: 19/240,511

(22) Filed: Jun. 17, 2025

Related U.S. Application Data
(63) Continuation-in-part of application No. PCT/KR2023/013006, filed on Aug. 31, 2023.

(30) Foreign Application Priority Data
Feb. 6, 2023 (KR) 10-2023-0015556
Aug. 30, 2023 (KR) 10-2023-0114453

(51) Int. Cl. G06N 20/00

(52) U.S. Cl. CPC G06N 20/00

(57) ABSTRACT

An electronic device according to an embodiment of the present invention is configured by including: a memory for storing an evaluation criterion related to ethicality of an artificial intelligence model performing a consultation; and a processor that measures an ethicality degree of result data output by the artificial intelligence model according to the evaluation criterion, wherein the ethicality degree is measured according to types of users performing consultations with the artificial intelligence model.

### CROSS-REFERENCE TO RELATED APPLICATIONS

This application is a continuation-in-part of International Application No. PCT/KR2023/013006, filed Aug. 31, 2023, which designated the U.S., and also claims the benefit of priority under 35 U.S.C. § 119(a) of Korean Patent Application No. 10-2023-0015556, filed on Feb. 6, 2023, and Korean Patent Application No. 10-2023-0114453, filed on Aug. 30, 2023, the contents of all of which are incorporated herein by reference in their entirety.

### TECHNICAL FIELD

The present disclosure relates to an apparatus for evaluating ethicality of an artificial intelligence (AI) model according to user types.

### BACKGROUND ART

With the rapid development of psychological consultation technology based on artificial intelligence, AI-based psychological consultation robots or apps are expected to greatly benefit the general public.

The AI-based psychological consultation technology has several advantages in terms of consultation fees and accessibility compared to traditional human counselor-based consultation methods. Accordingly, the AI-based psychological consultation technology may allow low-income users or users who are reluctant to disclose sensitive personal issues to more freely access psychological consultation.

However, since such AI-based psychological consultation technologies are implemented in the form of a black box, issues related to the ethicality of the output data may arise. Therefore, there is a need for further research on methods for implementing artificial intelligence models that provide user-friendly responses and explanations and evaluating the ethicality of the artificial intelligence models.

### DISCLOSURE

### Technical Problem

An embodiment of the present disclosure has been made to provide a consultation model customized to user characteristics based on explainable artificial intelligence (XAI).

An embodiment of the present disclosure has been made to classify consultation subjects into one of multiple types based on XAI and to evaluate the ethicality of an artificial intelligence model performing consultation according to user types.

The objectives to be achieved by the present disclosure are not limited to the above-mentioned objectives, and other objectives which are not mentioned will be more clearly understood by those skilled in the art from the following description and the embodiments of the present disclosure. Furthermore, it will be readily apparent that the objectives and advantages of the present disclosure can be achieved by the means and combinations thereof set forth in the claims.

### Technical Solution

To accomplish the above-mentioned objectives, according to the present disclosure, there is provided an electronic device including: a memory that stores evaluation criteria related to the ethicality of an artificial intelligence model performing consultation; and a processor that measures an ethicality degree of result data output by the artificial intelligence model according to the evaluation criteria, wherein the ethicality degree is measured according to user types of users consulting with the artificial intelligence model.

Moreover, the processor may include a user determination unit that measures scores for respective ethicality-related items of the user based on the consultation content between the artificial intelligence model and the user, and classifies the user by user types based on the item scores.

In this instance, the user types may be classified based on scores for items including disposition, virtue, personality, cognitive faculty, and personal environments. The disposition refers to a property regarding consistency, the virtue refers to a property regarding morality, the personality refers to a property regarding empathy, the cognitive faculty refers to a property regarding problem-solving ability, and the personal environments refer to a property related to social support.

Furthermore, the processor may include an ethicality determination unit measuring scores for respective ethicality-related items of the artificial intelligence model based on the consultation content between the artificial intelligence model and the user. The ethicality-related items of the artificial intelligence model may include interpretability, transparency, responsibility, bias, and stability.

The ethicality determination unit may measure the ethicality score of the artificial intelligence model based on a user's survey regarding results output by the artificial intelligence model or measure the ethicality score of the artificial intelligence model based on analysis of the user's condition changes after the consultation with the artificial intelligence model.

In another aspect of the present disclosure, a control method of an electronic device according to various embodiments of the present disclosure may include: performing consultation between an artificial intelligence model and a user; identifying the user type based on the consultation content; and measuring an ethicality degree of result data output by the artificial intelligence model according to pre-stored evaluation criteria, wherein the ethicality degree is measured according to the identified user type.

In another aspect of the present disclosure, a computer program stored in a computer-readable recording medium according to an embodiment of the present disclosure may be executed by a processor of an electronic device to perform the above control method.

### Advantageous Effect

According to an embodiment of the present disclosure, the apparatus for evaluating ethicality of an artificial intelligence model according to user types can evaluate the ethicality of the artificial intelligence model performing a consultation corresponding to user characteristics, thereby providing a user-customized consultation model.

Additionally, according to an embodiment of the present disclosure, the apparatus for evaluating ethicality of an artificial intelligence model according to user types can collect user personality data through a Q&A process with the explainable artificial intelligence (XAI)-based consultation model, thus performing user type identification in an environment similar to real consultations and reducing user fatigue without requiring a separate test.

In addition, according to an embodiment of the present disclosure, the apparatus for evaluating ethicality of an artificial intelligence model according to user types can perform consultation based on the explainable artificial intelligence (XAI) to provide human-friendly explanations of diagnostic results acceptable to consultation subjects and guidelines, thus improving user satisfaction.

### DESCRIPTION OF DRAWINGS

FIG. 1 is a diagram illustrating the configuration of an electronic device according to an embodiment of the present disclosure.

FIG. 2 is a diagram illustrating the configuration of a user determination unit according to an embodiment of the present disclosure.

FIG. 3 is a diagram illustrating the configuration of an ethicality determination unit according to an embodiment of the present disclosure.

FIG. 4 is a diagram for depicting an evaluation operation for output data of an artificial intelligence model according to user types according to an embodiment of the present disclosure.

FIGS. 5 and 6 are diagrams illustrating the evaluation operation of the artificial intelligence model of the electronic device according to an embodiment of the present disclosure.

### BEST MODE

According to an embodiment of the present disclosure, an electronic device may include a memory that stores evaluation criteria related to the ethicality of an artificial intelligence model performing consultation; and a processor that measures an ethicality degree of result data output by the artificial intelligence model according to the evaluation criteria, wherein the ethicality degree is measured according to user types of users consulting with the artificial intelligence model.

Moreover, the processor may include a user determination unit that measures scores for respective ethicality-related items of the user based on the consultation content between the artificial intelligence model and the user, and classifies the user by user types based on the item scores.

In this instance, the user types may be classified based on scores for items including disposition, virtue, personality, cognitive faculty, and personal environments. The disposition refers to a property regarding consistency, the virtue refers to a property regarding morality, the personality refers to a property regarding empathy, the cognitive faculty refers to a property regarding problem-solving ability, and the personal environments refer to a property related to social support.

Furthermore, the processor may include an ethicality determination unit measuring scores for respective ethicality-related items of the artificial intelligence model based on the consultation content between the artificial intelligence model and the user. The ethicality-related items of the artificial intelligence model may include interpretability, transparency, responsibility, bias, and stability.

The ethicality determination unit may measure the ethicality score of the artificial intelligence model based on a user's survey regarding results output by the artificial intelligence model or measure the ethicality score of the artificial intelligence model based on analysis of the user's condition changes after the consultation with the artificial intelligence model.

In another aspect of the present disclosure, a control method of an electronic device according to various embodiments of the present disclosure may include: performing consultation between an artificial intelligence model and a user; identifying the user type based on the consultation content; and measuring an ethicality degree of result data output by the artificial intelligence model according to pre-stored evaluation criteria, wherein the ethicality degree is measured according to the identified user type.

In another aspect of the present disclosure, a computer program stored in a computer-readable recording medium according to an embodiment of the present disclosure may be executed by a processor of an electronic device to perform the above control method.

### Mode for Invention

The above and other objectives, features, and advantages of the present disclosure will be easily understood from the following embodiments in conjunction with the accompanying drawings. However, the present disclosure may be embodied in different forms without being limited to the embodiments set forth herein. Rather, the embodiments disclosed herein are provided to make the disclosure thorough and complete and to sufficiently convey the spirit of the present disclosure to those skilled in the art, and the present disclosure is defined solely by the claims.

The terminology used herein is for the purpose of describing embodiments only and is not intended to be limiting. In the present disclosure, the singular forms are intended to include the plural forms as well, unless the context clearly indicates otherwise. The terms “comprises” and/or “comprising” when used in this specification do not preclude the presence or addition of one or more other components. The same reference numerals are used throughout the drawings to designate the same components, and the terms “and/or” specify the presence of stated components and a combination there of. It will be understood that, although the terms “first”, “second”, etc. may be used herein to describe various components, these components should not be limited by these terms. These terms are only used to distinguish one component from another component. Thus, a first component could be termed a second component without departing from the teachings of the present disclosure.

Unless otherwise defined, all terms including technical and scientific terms used herein have the same meaning as commonly understood by one of ordinary skill in the art to which the present disclosure belongs. Further, terms used herein will not be interpreted in an idealized or overly formal sense unless expressly so defined herein.

The term “unit” or “module” when used in the specification means a hardware component, such as a software, FPGA, or ASIC, and the “unit” or “module” performs certain roles. However, the “unit” or “module” is not limited to software or hardware. The “unit” or “module” may be configured to be present in an addressable storage medium and may be configured to cause one or more processors to perform operations. Thus, as an example, the “unit” or “module” includes components such as software components, object-oriented software components, class components and task components, as well as processes, functions, attributes, procedures, subroutines, segments of program code, drivers, firmware, microcode, circuitry, data, databases, data structures, tables, arrays and variables. The functions provided within the components and “units” or “modules” may be combined into a smaller number of components and “units” or “modules” or further separated into additional components and “units” or “modules”.

Spatially relative terms such as “below,” “beneath,” “lower,” “above,” and “upper” may be used to easily describe a correlation between one component and other components as shown in the drawings. The spatially relative terms should be understood as including different directions of components when in use or operation in addition to the directions depicted in the drawings. For example, when components depicted in the drawings are flipped, a component described as being “below” or “beneath” another component may then be positioned “above” the other component. Therefore, the exemplary term “below” may encompass both downward and upward directions. Components may also be oriented in different directions, and thus spatially relative terms may be interpreted based on such orientation.

According to an embodiment of the present disclosure, an electronic device 100 may store an artificial intelligence model (e.g., explainable artificial intelligence, XAI) that performs consultation for therapy and may evaluate the artificial intelligence model based on the content of the consultation performed using the artificial intelligence model.

Moreover, the terms “AI” or “artificial intelligence” may be regarded as referring to explainable Artificial Intelligence (XAI).”

Specific embodiments of control operations of the electronic device 100 will be described in detail with reference to the accompanying drawings below.

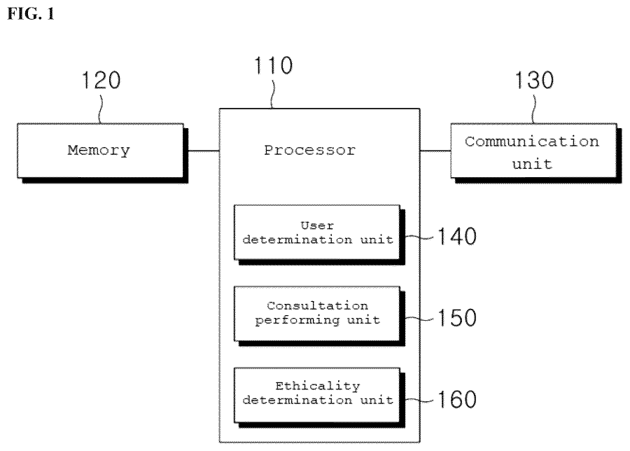

FIG. 1 is a diagram illustrating the configuration of the electronic device according to an embodiment of the present disclosure.

As illustrated in FIG. 1, the electronic device 100 according to an embodiment of the present disclosure may include a processor 110, a memory 120, and a communication unit 130.

Furthermore, the processor 110 may include a user determination unit 140, a consultation performing unit 150, and an ethicality determination unit 160.

Additionally, the memory 120 may include a conversation model based on artificial intelligence. Although not illustrated, the memory 120 may further include an artificial intelligence model (e.g., type determination model) for determining the user type, in addition to the conversation model.

As described above, the electronic device 100 may include various components. Hereinafter, each component will be described in more detail.

As described above, the processor 110 of the electronic device 100 may include the user determination unit 140, the consultation performing unit 150, and the ethicality determination unit 160.

The user determination unit 140 may perform all operations for determining the user type based on the personality evaluation theory.

The user determination unit 140 may perform an operation for determining the user type based on pre-stored evaluation criteria.

In this case, the operation for determining the user type may be performed based on an explainable artificial intelligence (XAI) model, and may classify the user into one of multiple types based on measured values for five evaluation items, which include the user's disposition, virtue, personality, cognitive faculty, and personal environments, measured from question-and-answer data obtained from users. The user type determined based on the measured values may include an avoidant type, a compromising type, or a problem-solving type.

To collect the response content required for determining the user type, the user determination unit 140 may request a predetermined amount of consultations to be conducted. When sufficient consultation data is acquired for identifying the user type, the user determination unit 140 may classify the user by types based on user response obtained during the consultation. The user determination unit 140 may categorize the user into one of three types (e.g., avoidant type, compromising type, problem-solving type) in accordance with the personality evaluation theory.

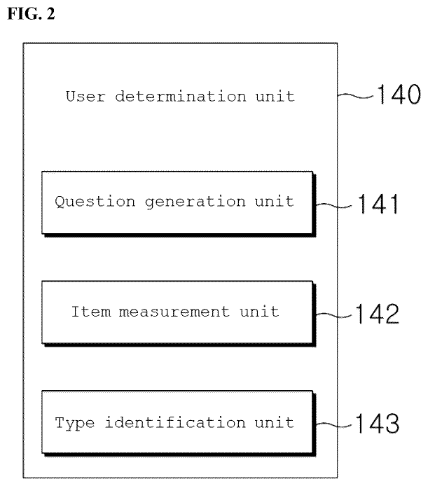

Further details of the user determination unit 140 are provided with reference to FIG. 2.

FIG. 2 is a diagram illustrating the configuration of the user determination unit according to an embodiment of the present disclosure.

As illustrated in FIG. 2, the user determination unit 140 may include a question generation unit 141, an item measurement unit 142, and a type identification unit 143.

First, the question generation unit 141 may generate questions required for determining the user type during an online consultation process between the artificial intelligence model and the user. Moreover, in various embodiments, the present disclosure may determine the user type through a separate user questionnaire outside the consultation process. Accordingly, the question generation unit 141 may generate questions required for conducting a user survey for type determination.

According to an embodiment, the question generation unit 141 may present an initial question identically to all users, regardless of who the user is. Alternatively, the initial question may be generated based on the user's basic personal information (e.g., gender, age, occupation, etc.). Without being limited to the above-described method, the question generation unit 141 may generate the initial question to be presented to the user who initiates the consultation in various ways.

After presenting the initial question to the user and receiving a response from the user, the question generation unit 141 may generate a follow-up question corresponding to the received response and present the follow-up question to the user.

In this instance, the question generation unit 141 may generate questions to measure the five evaluation items corresponding to the theory of personality evaluation (i.e., disposition, virtue, personality, cognitive faculty, and personal environments).

Specifically, a method for generating questions for measuring each of the five evaluation items is described as follows.

First, the question generation unit 141 may generate questions to measure “disposition” among the five evaluation items. The disposition is a latent variable that measures the strength and brightness/darkness of inherent temperament. The disposition is an item designed to evaluate a person's consistency by strength and weakness and activity level by brightness and darkness. For example, in the present disclosure, a user who is consistent and persistent with a bright personality may receive a high disposition score.

According to the nature of the “disposition” item, the question generation unit 141 may generate questions for evaluating the user's consistency, such as the will to follow through on planned activities. For example, the question generation unit 141 may generate questions such as: “Have you continued an exercise routine for more than one month?” or “Do people around you often describe you as a cheerful person?”, to measure the “disposition” item.

Next, the question generation unit 141 may generate questions to measure “virtue” among the five evaluation items. The “virtue” item is a variable that evaluates an individual's practical adherence to personal ethical standards and integrity (i.e., consistency between words and actions) constructed through personal efforts. A user who is deemed to possess ethical norms and integrity may receive a high virtue score.

According to the nature of the “disposition” item, the question generation unit 141 may generate questions such as: “Do you generally behave morally and in accordance with ethical principles without deceit?”, “Do you often prioritize appearances over humility and propriety?”, “Do you tend to maintain consistency between your words and actions?”, and “Do you often fail to demonstrate integrity and consistency in your words and actions?”.

Next, the question generation unit 141 may generate questions to measure “personality” among the five evaluation items. The “personality” item is designed to assess a user's empathy, tolerance (catholicity), and social adaptability. A user with high tolerance and strong social adaptability will receive a higher personality score.

According to the nature of the “personality” item, the question generation unit 141 may generate questions such as: “Do you usually demonstrate a high level of empathy and tolerance toward others?”, “Do you generally lack empathy and tolerance toward others?”, “Do you tend to get along well with people around you?”, and “Do you usually have difficulty in getting along with others?”.

Next, the question generation unit 141 may generate questions to measure “cognitive faculty” among the five evaluation items. The “cognitive faculty” item is a variable for evaluating an individual's problem-solving ability. A user with strong problem-solving abilities will receive a higher cognitive faculty score.

According to the nature of the “cognitive faculty” item, the question generation unit 141 may generate questions such as: “Do you make good use of your own experiential knowledge and advices of others?”, “Do you tend to rely on your own judgment, sometimes encountering difficulties in problem-solving?”, “Do you prefer rational and fundamental approaches to problem-solving?”, and “Do you prefer to rely on spontaneous ideas rather than rational and fundamental problem-solving methods?”

Lastly, the question generation unit 141 may generate questions to measure “personal environments” among the five evaluation items. The “personal environments” item is a variable that measures a user's social status and level of social activity. Additionally, the “personal environments” item is a variable that measure the degree of social support that the user receives from the user's peers and surrounding people, and social achieved status and social sphere in community activities. A user with high levels in social support or social achieved status and social sphere may receive a higher personal environment score.

According to the nature of the “personal environments” item, the question generation unit 141 may generate questions such as: “Are you generally respected and trusted by members of your organization or community?”, “Do you generally lack trust and recognition from members of your organization or community?”, “Are you usually active in helping your community and others?”, and “Are you generally inactive in helping your community or others?”

In addition, the question generation unit 141 may determine whether to generate additional questions based on the completion status of the evaluation for each item measured by the item measurement unit 142 and whether to change the topic of the questions. If the item measurement is not completed, the item measurement unit 142 may generate additional questions.

For instance, if the measurement of all five items is completed, the item measurement unit 142 may transmit a signal indicating completion to the question generation unit 141, then, the question generation unit 141 will not generate further questions and terminate the user type determination operation. On the other hand, if the item measurement unit 142 notifies that only four out of five items have been completed, the question generation unit 141 may control to change the topic of the subsequent questions.

Moreover, when the question generation unit 141 receives a response from the user, the question generation unit 141 may quantify the specificity of the response and determine the degree of detail for subsequent questions based on the quantified value.

The specificity of the user's response may be quantified, for example, by measuring the length of the response, measuring the variety of words used in the response, or by measuring both the length of the response and the variety of words.

The question generation unit 141 may generate additional questions reinforcing the degree of specificity when the obtained user response fails to meet a predefined threshold of specificity to be obtained.

For example, if the question generation unit 141 asks, “Do you generally follow through with your plans?” and receives a response with the response length below a certain threshold (e.g., fewer than 10 characters) such as “Yes” or “No” or the variety of words below a certain threshold (e.g., fewer than three distinct words), the question generation unit 141 may generate a more specific follow-up question on the same topic, such as “Have you ever successfully carried out a plan for over a month?”. Likewise, during the consultation process, if the received responses do not reach the target specificity level, the question generation unit 141 may continue generating additional questions with an enhanced specificity level.

The item measurement unit 142 may obtain user responses based on the questions provided to the user through the question generation unit 141 and measure the five evaluation items (disposition, virtue, personality, cognitive faculty, and personal environments) based on the personality evaluation theory according to the user's responses.

According to various embodiments, the item measurement unit 142 may input the user's responses into a separate artificial intelligence model to determine user types, and calculate values for each of the five evaluation items.

Next, a method of the item measurement unit 142 calculating a measurement value for each of the five items will be described. First, the process of calculating the measurement value for the “disposition” item by the item measurement unit 142 is as follows. The item measurement unit 142 may input the user's response, and then, calculate correlation values for the “consistency” and “activity level” which are sub-factors of the “disposition” item. In this instance, the artificial intelligence model may be designed such that the X-axis increases with the positive level of consistency (i.e., stronger consistency yields a higher value), and the Y-axis increases with the level of activity (i.e., higher energy and brightness yield a higher value).

According to various embodiments, the artificial intelligence model may be designed to assign each axis of the graph to a sub-factor of the five evaluation items and have an additional axis to record an association score between each item and user characteristics. Furthermore, the artificial intelligence model may calculate and record a stability score for the user's response to the additional axis (e.g., z-axis). The stability score may be a value determined based on a user response speed and consistency between the user's answers.

The type identification unit 143 may determine the user type as one of three predefined types based on the measurement values of the five evaluation items measured by the item measurement unit 142.

In this instance, the type identification unit 143 may classify the user as one of the three types according to the personality evaluation theory (e.g., avoidant type, compromising type, or problem-solving type). The avoidant type has the lowest level in motivation and willingness to solve problems, the problem-solving type exhibits the highest level in motivation and willingness to solve problems and means a person who is full of self-confidence. Additionally, the compromising type is an intermediate type that shows more initiative than the avoidant type but less than the problem-solving type.

Specifically, if the measurement values for all five evaluation items are below the respective thresholds, the type identification unit 143 may identify the user as the avoidant type. If the values for “disposition” and “cognitive faculty” among the five evaluation items are below the thresholds, the type identification unit 143 may identify the user as the compromising type. If the measurement values for all five evaluation items are above the thresholds, the type identification unit 143 may identify the user as the problem-solving type.

Among the components of the processor 110 illustrated in FIG. 1, the consultation performing unit 150 may perform the consultation operation for the user.

Specifically, the consultation performing unit 150 may support the consultation operation between the user and the artificial intelligence model.

According to an embodiment, the consultation performing unit 150 may perform the consultation under the support of the user determination unit 140. Before identifying the user type of a user, the consultation performing unit 150 may support the execution of conversation within a consultation screen to identify the user type under the control of the user determination unit 140.

Once the user type has been identified by the user determination unit 140, the consultation performing unit 150 may proceed with general consultation operations unrelated to type identification. For instance, the consultation performing unit 150 may perform various kinds of psychiatric consultation or personal problem-solving consultations.

Specifically, the consultation performing unit 150 may acquire consultation topics or psychiatric condition items requested by the user, and determine the scope or type of consultation depending on the acquired consultation topics or psychiatric condition items. For example, the consultation performing unit 150 may obtain information regarding a psychiatric condition item entered by the user (e.g., addiction), and determine to conduct a consultation to the user for addressing addiction-related issues depending on the obtained information.

Thereafter, when the scope or type of consultation has been determined, the consultation performing unit 150 may present questions to the user in order to proceed with the consultation, or may acquire conversation content initially input into a conversation screen by the user.

The consultation performing unit 150 may input the conversation content entered by the user into an explainable artificial intelligence (XAI) model, determine at least one corresponding response type to be provided to the user, and output consultation statements based on the determined response type.

According to an embodiment, the consultation performing unit 150 may perform human-friendly explanations (HFE) and perform interaction with the user based on the result of classifying the user type into at least three or more groups.

Specifically, according to various embodiments, the consultation performing unit 150 may output consultation content to be provided to the user (such as diagnostic results, identified user type information, and recommended consultation messages), and provide explanations for the reasoning of the consultation content.

The consultation performing unit 150 may classify the types of operations performed to output a setting mode during the consultation process and consultation results (e.g., consultation messages) generated based on the setting mode and provide explanations about the classification to the user. For example, if the consultation performing unit 150 provided the user with Solution A as a way to overcome a specific issue, the consultation performing unit 150 may present the rationale (information such as the identified personality type of the user) for outputting Solution A, including related materials.

Furthermore, the processor 110 may include an ethicality determination unit 160.

The ethicality determination unit 160 may evaluate and determine the ethicality and explainability of the artificial intelligence (XAI) model that performs the consultation, based on the conversation content identified during the consultation process with the user.

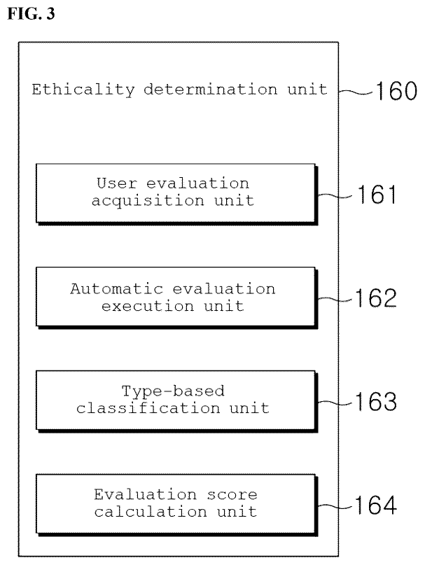

The ethicality determination unit 160 will be further described with reference to FIG. 3.

FIG. 3 is a diagram illustrating the configuration of the ethicality determination unit 160 according to an embodiment of the present disclosure.

As illustrated in FIG. 3, the ethicality determination unit 160 according to the embodiment of the present disclosure may include a user evaluation acquisition unit 161, an automatic evaluation execution unit 162, a type-based classification unit 163, and an evaluation score calculation unit 164.

First, the user evaluation acquisition unit 161 may, upon confirming that a predetermined amount of consultation has been conducted, request the user to evaluate the result data (consultation content) output to the user by the artificial intelligence model during the consultation operation.

For instance, the user evaluation acquisition unit 161 may request an evaluation from each user regarding each piece of result data output by the artificial intelligence model, or regarding the result data output by the artificial intelligence model for a specific conversation section or on a specific date.

Additionally, the user evaluation acquisition unit 161 may perform an evaluation of the ethicality of the artificial intelligence model based on the evaluation information provided by the user in response to the request.

Specifically, the evaluation operation requested to the user by the user evaluation acquisition unit 161 may be carried out by presenting to the user the items that serve as criteria for evaluating the ethicality of the artificial intelligence model and assigning a score for each individual item. In this instance, the evaluation items serving as criteria for the artificial intelligence model may include: comprehensibility (whether the decisions of the AI model are easily understandable by humans), fidelity (whether the decisions of the AI model are transparent, which may be validated through verification, testing, certification, etc.), responsibility (whether the AI model is designed to be responsible, including identify ability of responsibility and accountability among stakeholders), bias (whether the decisions of the AI model are fair, including bias in data collection and processing, algorithmic bias, accessibility, and fairness), and stability (whether the decisions of the AI model are trustworthy, including stability and usability).

As the user evaluation acquisition unit 161 directly presents the evaluation items to the user, the user may assign individual scores to the respective items, and the user evaluation acquisition unit 161 may acquire the scores for the respective items assigned by the user as evaluation information.

Meanwhile, the user evaluation acquisition unit 161 may conduct a conversation-based survey with the user for evaluating the ethicality of the artificial intelligence model, without directly presenting the evaluation items. In addition, the user evaluation acquisition unit 161 may analyze the user's responses collected during the process and automatically assign scores to the respective evaluation items based on the analysis results.

The automatic evaluation execution unit 162 may perform evaluation operations based on the consultation content collected during the consultation process between the user and the artificial intelligence model, rather than based on the user's survey responses. Accordingly, the automatic evaluation execution unit 162 may automatically generate an evaluation for each piece of result data output by the artificial intelligence model. However, the present disclosure is not limited thereto, and the automatic evaluation execution unit 162 may extract only a specific unit of the consultation content from the entire consultation process, based on criteria such as consultation content over a reference period (e.g., one day), reference conversation unit (e.g., six conversations), or consultation content by topic, and may perform an evaluation of the result data output by the artificial intelligence model based on the extracted content.

In this case, according to various embodiments, the automatic evaluation execution unit 162 may extract only the content output by the artificial intelligence model from the conversation content during the consultation and may measure a score for at least one evaluation item based on the content. For instance, the automatic evaluation execution unit 162 may determine whether the content output by the artificial intelligence model is true or not and, in response, assign a score for the evaluation item corresponding to stability.

In addition, the automatic evaluation execution unit 162 may analyze changes in the user observed during the consultation process, and based on the analysis, measure scores for at least one evaluation item. For instance, the automatic evaluation execution unit 162 may identify the occurrence of negative changes in the user during the consultation process (e.g., increase in the frequency of negative expressions, increase in the intensity of negative expressions, decrease in the length of responses beyond a predetermined value), positive changes (e.g., increase in the frequency of positive expressions, increase in the intensity of positive expressions, increase in the specificity of responses, increase in the response length beyond a predetermined value), or changes in interest toward a specific direction (e.g., increase in political bias, increase in prejudice against a specific group), and measure scores for each evaluation item corresponding to each situation. Moreover, in this case, the automatic evaluation execution unit 162 may identify not only overall negative or positive changes in the user after the consultation, but also the occurrence of negative or positive changes in specific fields, and measure scores for each evaluation item corresponding to each situation.

For example, if the automatic evaluation execution unit 162 determines that the user's statements increasingly show political bias, the automatic evaluation execution unit 162 may reduce the score for the bias evaluation item. Alternatively, if the automatic evaluation execution unit 162 identifies overall negative change in the user, the automatic evaluation execution unit 162 may reduce the score for the stability evaluation item.

In addition, if the automatic evaluation execution unit 162 determines that the number of times the user re-questions the intention in relation to the output result from the artificial intelligence model exceeds a certain threshold, the automatic evaluation execution unit 162 may reduce the score for comprehensibility from a default value. For instance, after the artificial intelligence model outputs an imperative or suggestive sentence, if it is confirmed that the number of the user's questions including a word (predefined) to confirm reason such as “why” exceeds a certain threshold, the automatic evaluation execution unit 162 may determine that the user is questioning the intention of the content output by the artificial intelligence model. Furthermore, if such intention questions are determined to exceed the reference count, the automatic evaluation execution unit 162 may evaluate that the results output by the artificial intelligence model has low comprehensibility. Through the evaluation methods, the automatic evaluation execution unit 162 may reduce the score for the comprehensibility evaluation item by a reference value (e.g., one star) if the number of intention questions from the user's statement exceeds a threshold (e.g., three times). Additionally, as the number of such intention questions exceeds the threshold, the score for the comprehensibility evaluation item may be further reduced in proportion to the increase in the number of intention questions. Accordingly, the automatic evaluation execution unit 162 may not reflect intention questions in the comprehensibility evaluation unless the intention questions reach the reference count.

Additionally, the automatic evaluation execution unit 162 may analyze the response method output by the artificial intelligence model following the input of a problematic statement by the user, and perform an evaluation operation based on the analysis. For example, the automatic evaluation unit 162 may analyze the response output by the artificial intelligence model in response to the input to evaluate whether an attempt was made to correct a problematic statement (e.g., illegal content, prohibited words such as profanity, or expressions indicating suicidal thoughts) entered by a user, and to what extent such correction was made, and then, based on the analysis, may assign a score regarding the accountability or stability of the artificial intelligence model. In this case, the automatic evaluation unit 162 may determine whether a consultation phrase opposite in meaning to the problematic statement input by the user was output, in order to determine whether a corrective attempt was made.

The type-based classification unit 163 may classify the evaluation information obtained by the user evaluation acquisition unit 161 and the automatic evaluation unit 162 according to the type of the user receiving the consultation. In this instance, the user type refers to the type identified by the user determination unit 140.

According to an embodiment of the present disclosure, since the user determination unit 140 classifies the users into three types, the evaluation information may be classified into three categories based on the user types.

The evaluation score calculation unit 164 may calculate an evaluation score for the artificial intelligence model for each user type based on the evaluation information classified into three categories by the type-based classification unit 163.

In this case, the evaluation operation may be performed not only on the artificial intelligence model, but also on each piece of output data (e.g., individual solution) or a specific unit of consultation content output by the artificial intelligence model. Accordingly, evaluation scores may also be calculated for each piece of output data or for each unit of consultation content.

The evaluation results of the artificial intelligence model may be used to improve the artificial intelligence model in the future. Furthermore, the evaluation results of the artificial intelligence model evaluated by user types may be used to generate a consultation model specialized for each user type.

According to various embodiments, the evaluation score calculation unit 164 may determine consultation topics and fields in which the evaluation score deviation by user types is greater than the reference value, and topics and fields in which the evaluation score deviation is less than the reference value. Moreover, in consultation topics and fields in which the evaluation score deviation by user types is greater than the reference value, the evaluation score calculation unit 164 may identify the result data (or the corresponding type) of the artificial intelligence model that received high evaluation (e.g., exceeding the reference score) for each user type, and support the consultation performing unit 150 to apply the identified result data in future consultations.

Accordingly, when a consultation is conducted in consultation topics and fields in which the evaluation score deviation by user types is greater than the reference value, the consultation performing unit 150 may apply the artificial intelligence model (or corresponding result data type of the artificial intelligence model) that received high evaluation for the identified user type after previously identifying the user type. As a result, even if a certain response from the artificial intelligence model may induce negative changes or be perceived as untrustworthy for other user types, if the response is evaluated to induce positive changes or be trustworthy for the identified user type, the consultation performing unit 150 may control the corresponding response to be output in the consultation process for the corresponding user.

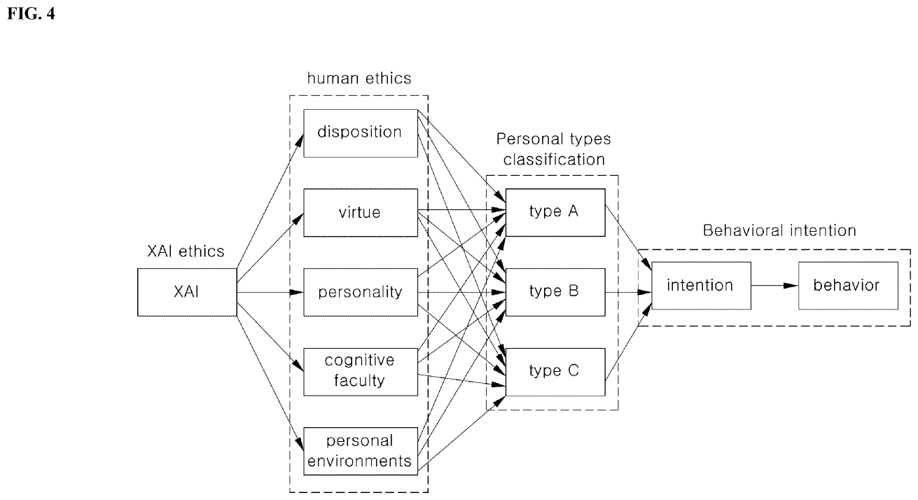

FIG. 4 is a diagram for depicting an evaluation operation for output data of an artificial intelligence model according to user types according to an embodiment of the present disclosure.

As illustrated in FIG. 4, the artificial intelligence (XAI) model that performs consultation or the result data output by the artificial intelligence model according to an embodiment of the present disclosure may be evaluated for the ethicality or explain ability of intention based on three user types classified according to scores in five evaluation items for the user.

As a reference, the memory 120, which is one of the components of the electronic device 100 of FIG. 1, may store commands and algorithms required to perform overall operations according to an embodiment of the present disclosure. The memory 120 may store artificial intelligence models necessary for identifying the user type and conducting consultation with the user based on the identified type.

The communication unit 130 may support communication operations with various user terminals for acquiring user input. For instance, the communication unit 130 may receive voice data or text data for consultation with the user via a separate user terminal. Furthermore, the communication unit 130 may receive evaluation data from the user terminal regarding the artificial intelligence model or the result data output by the artificial intelligence model.

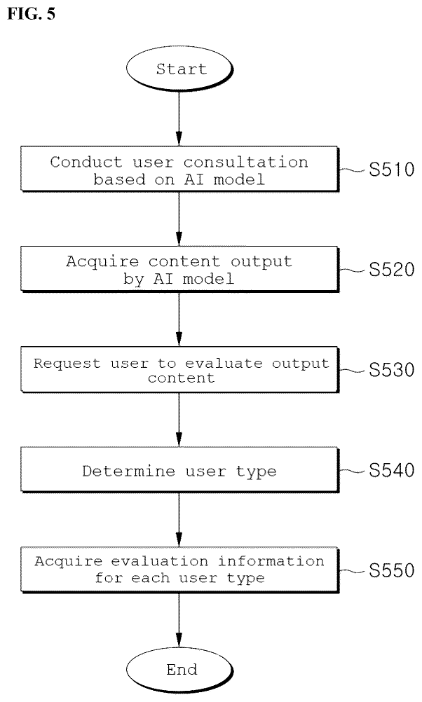

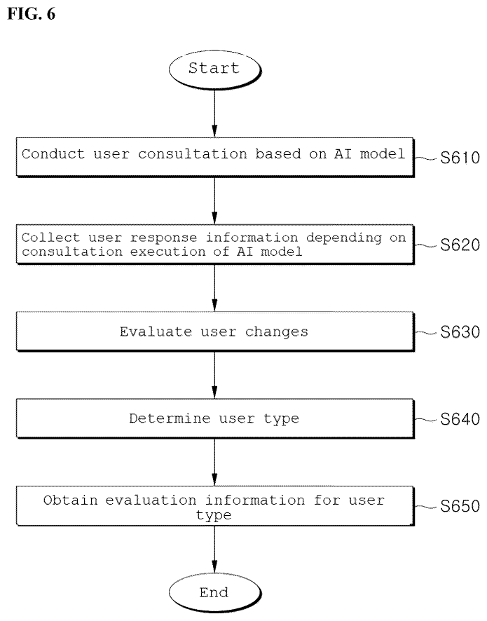

FIGS. 5 and 6 are diagrams illustrating the evaluation operation of the artificial intelligence model of the electronic device according to an embodiment of the present disclosure.

FIG. 5 illustrates a method of evaluating the artificial intelligence model based on content directly assessed by the user after confirming the output result of the model, and FIG. 6 illustrates a method of evaluating the artificial intelligence model based on user changes identified through user responses during the consultation process.

Referring to each drawing, the methods will be described in detail.

As illustrated in FIG. 5, the electronic device 100 according to an embodiment of the present disclosure may perform operation S510 for conducting a user consultation based on the artificial intelligence model.

Subsequently, the electronic device 100 may perform operation S520 for acquiring content output by the artificial intelligence model during the consultation.

Then, the electronic device 100 may perform operation S530 of requesting the user to evaluate the content output by the artificial intelligence model.

Thereafter, the electronic device 100 may perform operation S540 of determining the user type according to preset evaluation criteria.

Subsequently, the electronic device 100 may perform operation S550 of acquiring evaluation information for each user type. In this instance, the operation S540 of determining the user type may be performed at any point from the beginning of the consultation using the artificial intelligence model to a point before the generation of evaluation information for each user type.

FIG. 6 illustrates a sequence of operations in which the evaluation of output results from an artificial intelligence model is automatically performed based on the consultation content, without relying on a user's evaluation.

As illustrated in FIG. 6, after performing operation S610 in which the AI model-based consultation is conducted, the electronic device 100 according to an embodiment of the present disclosure may proceed to operation S620 to collect consultation content and user response information generated during the consultation process.

Subsequently, the electronic device 100 may perform operation S630 to evaluate user changes based on the collected consultation content. In this instance, the user changes may include, for example, an increase in positivity or negativity in a specific field of the user.

In addition, the electronic device 100 may perform operation S640 to determine the user type through the consultation content. As described with reference to FIG. 5, the timing of the operation for determining the user type may vary.

Afterward, the electronic device 100 may perform operation S650 to obtain evaluation information for each user type.

In summary, the electronic device according to an embodiment of the present disclosure may include: a memory that stores evaluation criteria related to the ethicality of an artificial intelligence model performing consultation; and a processor that measures an ethicality degree of result data output by the artificial intelligence model according to the evaluation criteria, wherein the ethicality degree is measured according to user types of users consulting with the artificial intelligence model.

Moreover, the processor may include a user determination unit that measures scores for respective ethicality-related items of the user based on the consultation content between the artificial intelligence model and the user, and classifies the user by user types based on the item scores.

In this instance, the user types may be classified based on scores for items including disposition, virtue, personality, cognitive faculty, and personal environments. The disposition refers to a property regarding consistency, the virtue refers to a property regarding morality, the personality refers to a property regarding empathy, the cognitive faculty refers to a property regarding problem-solving ability, and the personal environments refer to a property related to social support.

Furthermore, the processor may include an ethicality determination unit measuring scores for respective ethicality-related items of the artificial intelligence model based on the consultation content between the artificial intelligence model and the user. The ethicality-related items of the artificial intelligence model may include interpretability, transparency, responsibility, bias, and stability.

Additionally, the ethicality determination unit may measure the ethicality score of the artificial intelligence model based on a user's survey regarding results output by the artificial intelligence model or measure the ethicality score of the artificial intelligence model based on analysis of the user's condition changes after the consultation with the artificial intelligence model.

In another aspect of the present disclosure, a control method of an electronic device according to various embodiments of the present disclosure may include: performing consultation between an artificial intelligence model and a user; identifying the user type based on the consultation content; and measuring an ethicality degree of result data output by the artificial intelligence model according to pre-stored evaluation criteria, wherein the ethicality degree is measured according to the identified user type.

In another aspect of the present disclosure, a computer program stored in a computer-readable recording medium according to an embodiment of the present disclosure may be executed by a processor of an electronic device to perform the above control method.

The ethicality evaluation operation performed by the electronic device 100 according to various embodiments of the present disclosure may be applied not only in psychological consultation but also in various fields such as efficient learning, advanced AI-based decision-making (cognition, judgment, reasoning), trustworthy and safe AI, and industrial applications of AI.

Moreover, according to various embodiments, each item of the explainable artificial intelligence (XAI) in the artificial intelligence model for ethicality evaluation may be designed to influence the ethical characteristics of the user.

Furthermore, according to various embodiments, the electronic device 100 may support users in evaluating the ethicality scores of the artificial intelligence model.

Additionally, according to various embodiments, the electronic device 100 may determine the importance of ethicality items in the artificial intelligence model to vary depending on the user type. In other words, in the ethicality evaluation method of the artificial intelligence model, the ethicality items that are considered important may differ depending on the characteristics of the user.

According to various embodiments, during the consultation operation, the electronic device 100 may conduct consultation not only based on the user's psychological characteristics but also based on a multidimensional evaluation including morality, problem-solving ability, and social behavior.

According to various embodiments, the electronic device 100 may identify artificial intelligence models with relatively high ethicality evaluation scores (i.e., high explain ability). The artificial intelligence models with high scores may promote more active user engagement than black-box type models and may also enhance the effectiveness of the consultation.

According to an embodiment of the present disclosure, the electronic device 100 may include a processor 110, a memory 120, and a communication unit 130.

The memory 120 may store various programs and data necessary for the operation of the electronic device. The memory 120 may be implemented as non-volatile memory, volatile memory, flash memory, a hard disk drive (HDD), or a solid state drive (SSD).

The communication unit 130 may perform communication with an external device. In particular, the communication unit 130 may include various communication chips such as a Wi-Fi chip, a Bluetooth chip, a wireless communication chip, an NFC chip, and a low-power Bluetooth chip (BLE chip). At this time, the Wi-Fi chip, Bluetooth chip, and NFC chip communicate in the LAN mode, Wi-Fi mode, Bluetooth mode, and NFC mode, respectively. When using the Wi-Fi chip or Bluetooth chip, various pieces of connection information such as SSID and session key are first transmitted and received. After establishing a communication link using this information, various pieces of information may be sent and received. The wireless communication chip refers to a chip that performs communication according to various communication standards such as IEEE, Zigbee, 3G (3rd Generation), 3GPP (3rd Generation Partnership Project), and LTE (Long Term Evolution).

The processor 110 may control the overall operation of the user device using various programs stored in the memory 120. The processor may include RAM, ROM, a graphics processing unit, a main CPU, first to nth interfaces, and buses. At this time, the RAM, ROM, graphics processing unit, main CPU, first to nth interfaces, etc. may be connected to each other via a bus.

The RAM stores O/S and application programs. Specifically, when the electronic device boots up, the O/S may be stored in the RAM, and various application data selected by the user may be stored in the RAM.

The ROM stores a command set for system booting, etc. When a turn-on command is input and power is supplied, the main CPU copies the O/S stored in the memory 200 to the RAM according to the command stored in the ROM and executes the O/S to boot the system. When booting is complete, the main CPU copies various application programs stored in the memory 200 to the RAM and executes the application programs copied to the RAM to perform various operations.

The main CPU accesses the memory 120 and performs booting using the OS stored in the memory 120. Further, the main CPU performs various operations using various programs, contents, data, etc. stored in the memory 120.

The first to n interfaces are connected to the various components described above. One of the first to n interfaces may be a network interface connected to the external device via a network.

Meanwhile, the processor may control the artificial intelligence model. In this case, it goes without saying that a control unit may include a graphics-dedicated processor (e.g., GPU) for controlling the artificial intelligence model.

The processor 110 may include one or more cores (not shown) and a graphics processing unit (not shown) and/or a connection path (e.g., a bus, etc.) for transmitting and receiving signals with other components.

The processor according to an embodiment performs a method described in connection with the present disclosure by executing one or more instructions stored in the memory.

Meanwhile, the processor 110 may further include RAM (Random Access Memory, not shown) and ROM (Read-Only Memory, not shown) for temporarily and/or permanently storing a signal (or data) processed within the processor. Further, the processor may be implemented in the form of a system on chip (SoC), which includes at least one of a graphics processing unit, RAM, and ROM.

The memory 120 may store programs (one or more instructions) for processing and controlling the processor 110. Programs stored in the storage may be divided into multiple modules according to their functions.

The steps of the method or algorithm described in connection with an embodiment of the present disclosure may be implemented directly in hardware, in a software module executed by hardware, or through a combination thereof. The software module may reside in a random access memory (RAM), a read only memory (ROM), an erasable programmable ROM (EPROM), an electrically erasable programmable ROM (EEPROM), a flash memory, a hard disk, a removable disk, a CD-ROM, or any other form of computer readable recording medium that is well known in the art to which the present disclosure pertains.

The components of the present disclosure can be implemented as a program (or application) to be executed in combination with a computer, which is hardware, and stored in a medium. The components of the present disclosure may be implemented by software programming or software elements. Similarly, the embodiment may be implemented in a programming or scripting language, such as C, C++, Java, or assembler, including various algorithms implemented as a combination of data structures, processes, routines, or other programming constructs. The functional aspects may be implemented as algorithms executed on one or more processors 100.

Although the present disclosure has been described above with reference to preferred embodiments, it will be understood by those skilled in the art that various modifications and changes may be made to the present disclosure without departing from the scope of the present disclosure. It should be noted that in order to achieve the intended effect of the present disclosure, it is not necessary to separately include all the functional blocks illustrated in the drawings or follow all the sequences illustrated in the drawings in the exact order depicted, and even if not, it may fall within the technical scope of the present disclosure described in the claims.

1. An electronic device comprising:
a memory that stores evaluation criteria related to the ethicality of an artificial intelligence model performing consultation; and
a processor that measures an ethicality degree of result data output by the artificial intelligence model according to the evaluation criteria, wherein the ethicality degree is measured according to user types of users consulting with the artificial intelligence model.

2. The electronic device according to claim 1, wherein the processor includes a user determination unit that measures scores for respective ethicality-related items of the user based on the consultation content between the artificial intelligence model and the user, and classifies the user into a user type based on the item scores.

3. The electronic device according to claim 2, wherein the user types are classified based on scores for items including disposition, virtue, personality, cognitive faculty, and personal environments.

4. The electronic device according to claim 1, wherein the processor includes an ethicality determination unit measuring scores for respective ethicality-related items of the artificial intelligence model based on the consultation content between the artificial intelligence model and the user, and
wherein the ethicality-related items of the artificial intelligence model may include interpretability, transparency, responsibility, bias, and stability.

5. The electronic device according to claim 4, wherein the ethicality determination unit measures the ethicality score of the artificial intelligence model based on a user's survey regarding results output by the artificial intelligence model.

6. The electronic device according to claim 4, wherein the ethicality determination unit measures the ethicality score of the artificial intelligence model based on analysis of the user's condition changes after the consultation with the artificial intelligence model.

7. A control method of an electronic device comprising the steps of:
performing consultation between an artificial intelligence model and a user;
identifying the user type based on the consultation content; and
measuring an ethicality degree of result data output by the artificial intelligence model according to pre-stored evaluation criteria, wherein the ethicality degree is measured according to the identified user type.

8. A computer program stored in a computer-readable recording medium which is executed by a processor of an electronic device to perform the control method according to claim 7.
